(19) United States
(12) Patent Application Publication
(10) Pub. No.: US 2021/0146074 A1
(43) Pub. Date: May 20, 2021

(54) RESUSCITATION BAG WITH PEP EXHAUST VALVE COMPATIBLE WITH THORACIC COMPRESSIONS

(71) Applicants: Air Liquide Santé (International), Paris (FR); Air Liquide Medical Systems, Antony (FR)

(72) Inventors: THierry BOULANGER, Media, PA (US); Jean-Christophe RICHARD, Antony (FR); Marceau RIGOLLOT, Montrouge (FR); Jean-Marc GINER, Puteaux (FR)

(21) Appl. No.: 16/621,562

(22) PCT Filed: Nov. 30, 2017
(86) PCT No.: PCT/EP2017/080969

§ 371 Date: Dec. 11, 2019

Related U.S. Application Data
(60) Provisional application No. 62/525,399, filed on Jun. 27, 2017.

(51) Int. Cl. A61M 16/00; A61M 16/20

(52) U.S. Cl. CPC A61M 16/0078; A61M 16/209; A61M 16/201; A61M 16/0084

(57) ABSTRACT

The invention concerns an artificial resuscitation bag (5) comprising a deformable bag (54) comprising a gas inlet (54A) and a gas outlet (54B), a gas reservoir (59) comprising an outlet orifice (59A), a first conduit element (56) fluidly connected to the outlet orifice (59A) of the gas reservoir (59) and to the gas inlet (54A) of the deformable bag (54), a first one-way admission valve (57) arranged in the first conduit element (56) and fluidly communicating with the ambient atmosphere for allowing ambient air to enter into the first conduit element (56), and a second one-way valve (55) arranged in the first conduit element (56) between the first one-way admission valve (57) and the gas inlet (54A) of the deformable bag (54) for allowing gas to travel only from the first conduit element (56) to the deformable bag (54).

### CROSS REFERENCE TO RELATED APPLICATION

This application claims the benefit of priority to U.S. Provisional Patent Application No. 62/525,399, filed Jun. 27, 2017, the entire contents of which are incorporated herein by reference.

### BACKGROUND

The present invention relates to an artificial respiration device, namely an artificial resuscitation bag that can be used for resuscitating a person, i.e. a patient, in state of cardiac arrest, and an installation comprising such an artificial resuscitation bag for resuscitating a person in state of cardiac arrest.

Cardiac arrest is a condition affecting hundreds of thousand people every year with a very poor prognosis.

One of the main lifesaving actions is to apply thoracic compressions or ‘TCs’ along with brief intervals of lung ventilation with a resuscitation bag. TCs are successive compressions and decompressions exerted on the thoracic cage of the person, i.e. the patient, in cardiac arrest. TCs aim at partially restoring inhalation and exhalation phases and therefore gas exchanges in the lungs, as well as promoting or restoring blood circulation toward the organs and especially the brain of the patient.

As these compressions and decompressions only mobilize small volumes of gas in and out of the patient's airways, it is advocated to perform regularly further gas insufflations to bring fresh O₂-containing gas into the lungs thereby enhancing the gas exchanges.

Usually, fresh O₂-containing gas is delivered by a resuscitation bag linked with an oxygen source and connected to the patient through a respiratory interface, typically a facial mask, a laryngeal mask, or an endotracheal tube.

To date, it is recommended to interpose 2 insufflations every 30 chest compressions, whereas the ideal rate of compressions, according to international guidelines, is between 100 and 120 compressions per minute (c/min).

However, several studies have shown that it is difficult for rescuers to correctly perform the resuscitation sequence and that the interruptions of TCs to initiate the insufflations with a resuscitation bag are often too long and deleterious with respect to the patient's outcome, as rapidly affecting the hemodynamic, i.e., in other words, offsetting the benefits of the TCs themselves.

Some respiratory assistance devices have been proposed for overcoming the drawbacks associated with resuscitation bags. Among them, the most popular are Continuous Positive Airway Pressure apparatus, also called “CPAP systems” or “CPAP devices”, that rely on an oxygen containing-gas supply, at a pressure above 1 atm, for creating a continuous positive pressure at the patient's airways depending on the continuous flow of oxygen (e.g. the higher the oxygen flow, the higher the positive pressure).

During thoracic compressions/decompressions, small volumes are flowing in and out of the patient's airways at a positive pressure which helps keep the alveoli of the lungs open thereby promoting and/or enhancing gas exchanges. In addition, the positive pressure creates a resistance to gas expulsion during the TC phases, which improves the energy transmission to the heart thereby promoting a better cardiac output.

However, if these CPAP systems have demonstrated to be beneficial over basic TCs, e.g. without an extra respiratory assistance device, and could represent an interesting alternative, there is still room for improvement as small volumes are still mobilized during the TCs and intermittent insufflations with a resuscitation bag would be beneficial to bring further fresh O₂-containing gas into the lungs and thereby improve CO₂ clearance.

Unfortunately, such CPAP systems cannot function with current resuscitation bags without threatening the patient's life, due to serious adverse events that can be caused by the design of the CPAP systems themselves (especially from the continuous flow of oxygen that is supposed to set the positive pressure of the CPAP system).

### SUMMARY

A main goal of the present invention is to fix the problem encountered with current resuscitation bags, in particular to provide an improved resuscitation bag allowing continuous TCs and, when required, enabling insufflations of given volumes of fresh O₂-containing gas, while keeping a continuous positive pressure of gas into the patient's lungs, without the need of any CPAP systems.

A solution according to the present invention concerns an artificial resuscitation bag comprising:
a deformable bag comprising a gas inlet and a gas outlet,
a gas reservoir comprising an outlet orifice,
a first conduit element fluidly connected to the outlet orifice of the gas reservoir and to the gas inlet of the deformable bag,
a first one-way admission valve arranged in the first conduit element and fluidly communicating with the ambient atmosphere for allowing ambient air to enter into the first conduit element, and
a second one-way valve arranged in the first conduit element between the first one-way admission valve and the gas inlet of the deformable bag for allowing the gas to travel only from the first conduit element to the deformable bag,

and further comprising a first PEP exhaust valve arranged in the first conduit element and fluidly communicating with the ambient atmosphere for venting gas to the atmosphere when the gas pressure, into the first conduit element, exceeds a given threshold.

Depending on the embodiment, an artificial resuscitation bag according to the present invention can comprise of one or several of the following additional features:
the opening pressure of PEP exhaust valve is of at least 1 cmH₂O.
the opening pressure of PEP exhaust valve is of between 1 cmH₂O and 30 cmH₂O, preferably of at least about 5 cmH₂O.
the first PEP exhaust valve comprises a spring and a membrane, said spring applying a constant force on the membrane corresponding to the threshold pressure.
the first PEP exhaust valve comprises an inlet port in fluid communication with the first conduit.
the first conduit element comprises an oxygen entry arranged between the outlet orifice of the gas reservoir and the second one-way valve.
the first conduit element comprises an inner passage for the gas.
it comprises a gas conduit in fluid communication with the gas outlet of the deformable bag.
it comprises an overpressure valve arranged in the gas conduit in fluid communication with the gas outlet of the deformable bag.
it comprises a third one-way valve arranged in the gas conduit downstream of the overpressure valve.
it comprises further a pneumatic control valve arranged in the gas conduit downstream of the third one-way valve.
the pneumatic control valve comprises a deformable membrane.
it comprises a derivation conduct having a first end fluidly connected to the gas conduit, between the gas outlet of the deformable bag and the overpressure valve, and a second end fluidly connected to the inner compartment of the pneumatic control valve.
it comprises a gas delivery conduit in fluid communication with the gas conduit for conveying at least part of the gas circulating into the gas conduit to a patient interface.
the patient interface comprises of a respiratory mask or a tracheal cannula.
the gas conduit conveys at least a part of the gas exiting the deformable bag through the gas outlet.
the overpressure valve is configured to vent to the atmosphere at least part of the gas present in the gas conduit, when the gas pressure in the gas conduit exceeds a given value.
the artificial resuscitation bag further comprises of a second one-way valve arranged in a conduit in fluid communication with the gas inlet of the deformable bag.
a third one-way valve is arranged in the gas conduit and configured for allowing a circulation of gas in the gas conduit only in the direction from the deformable bag toward the pneumatic control valve.
it further comprises an oxygen line fluidly connected to the first conduit.
it further comprises an oxygen distribution system comprising a gas distributor and a by-pass line connected to said gas distributor.
the gas distributor is arranged on the oxygen line.
the by-pass line is fluidly connected to the gas distributor and to the patient interface.

Further, the present invention also concerns an installation for resuscitating a person in state of cardiac arrest comprising:
an artificial resuscitation bag according to the present invention, and
an O₂ source fluidly connected to the artificial resuscitation bag by means of an oxygen line, for providing oxygen to said artificial resuscitation bag.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 represents an embodiment of the resuscitation bag according to the prior art.

FIG. 2 illustrates an embodiment of the resuscitation bag according to the prior art.

FIG. 3A illustrates an embodiment of the resuscitation bag according to the prior art.

FIG. 3B illustrates an embodiment of the resuscitation bag according to the prior art.

FIG. 4 illustrates an embodiment of the resuscitation bag according to the prior art.

FIG. 5 illustrates an embodiment of the resuscitation bag according to the prior art.

FIG. 6 illustrates an embodiment of the resuscitation bag according to the prior art.

FIG. 7A illustrates an embodiment of the resuscitation bag according to the present invention.

FIG. 7B illustrates an embodiment of the resuscitation bag according to the present invention.

FIG. 7C illustrates an embodiment of the resuscitation bag according to the present invention.

FIG. 8 illustrates an embodiment of the resuscitation bag according to the present invention.

FIG. 9 illustrates an embodiment of the resuscitation bag according to the present invention.

FIG. 10 illustrates an embodiment of the resuscitation bag according to the present invention.

FIG. 11 illustrates an embodiment of the resuscitation bag according to the present invention.

FIG. 12 is another embodiment of the resuscitation bag according to the present invention.

FIG. 13A illustrates an embodiment of a pneumatic control valve of a resuscitation bag according to the present invention.

FIG. 13B illustrates an embodiment of a pneumatic control valve of a resuscitation bag according to the present invention.

### DESCRIPTION OF PREFERRED EMBODIMENTS

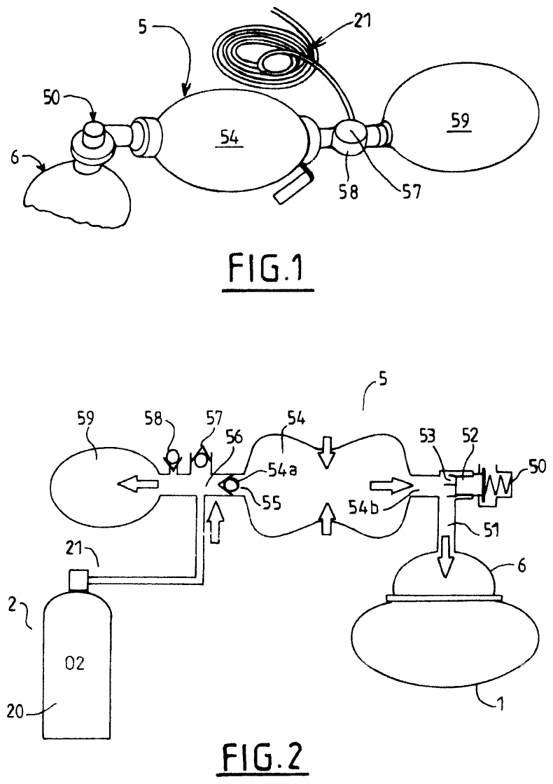

FIGS. 1 and 2 show a commercially available resuscitation bag 5 comprising of a respiratory interface 6 for feeding a gas to a patient, typically a respiratory mask, a flexible bag 54, and a valve element 50 for diverting the gas in and out of the patient, during insufflation and exsufflation phases, and a source of an oxygen-containing gas 2, such as or including a gas cylinder 20 containing oxygen, which is delivered during insufflation phases.

The flexible bag 54 is filled with fresh gas formed by a mixture of oxygen provided by an oxygen line 21 connected to the oxygen source 2 (cf. FIG. 2), typically a medical grade oxygen cylinder 20, and ambient air provided by an admission valve 57 in fluid communication with the ambient atmosphere.

A supplementary gas reservoir 59 can be added to increase the availability of oxygen. Further, a first exhaust valve 58 is provided for venting gas in the case of overpressure.

In FIG. 2, a patient 1 is connected to the resuscitation bag 5, via a respiratory interface 6, e.g. a facial mask, a laryngeal mask or similar.

The oxygen source 2, typically a cylinder 20 of medical grade oxygen, is fluidly connected via an oxygen line or tubing 21 and a first conduit element 56, to the flexible bag 54, the tubing 21 being fluidly connected to the first conduit element 56. The first conduit element 56 is further fluidly communicating with the inlet orifice 54A of the flexible bag 54.

When the operator squeezes the flexible bag 54 to perform an insufflation of gas to the patient 1, the flow of gas exiting the flexible bag 54 through its outlet orifice 54B travels to the patient 1 into the lumen of a second conduit 51 that is fluidly connected to the respiratory interface 6, such as a facial mask. At the same time, the flow of gas exiting the flexible bag 54 occludes the exhalation port 52 of a third exhaust valve 53 that is arranged in the second conduit 51, i.e. downstream of gas bag 54, as shown in FIG. 2.

This generates positive pressure which, as a result, forces second one-way valve 55 arranged upstream of the bag 54 to close thereby preventing the gas of bag 54 to flow backward, i.e. in the first conduit element 56, and to escape via the first exhaust valve 58. Meanwhile, the flow of oxygen travelling in tubing 21 enters into the first conduit element 56 and fills the supplementary reservoir 59 that is fluidly connected to first conduit element 56.

Due to the slightly positive pressure in first conduit element 56, the air admission valve 57 is closed. In the case where the reservoir 59 becomes over-distended by the entering flow of gas, a pressure increase will occur in first conduit element 56 and the gas in excess will be vented to the ambient atmosphere by the first exhaust valve 58. The opening pressure of the first exhaust valve 58 is close to 0, but slightly positive due to mechanical frictions.

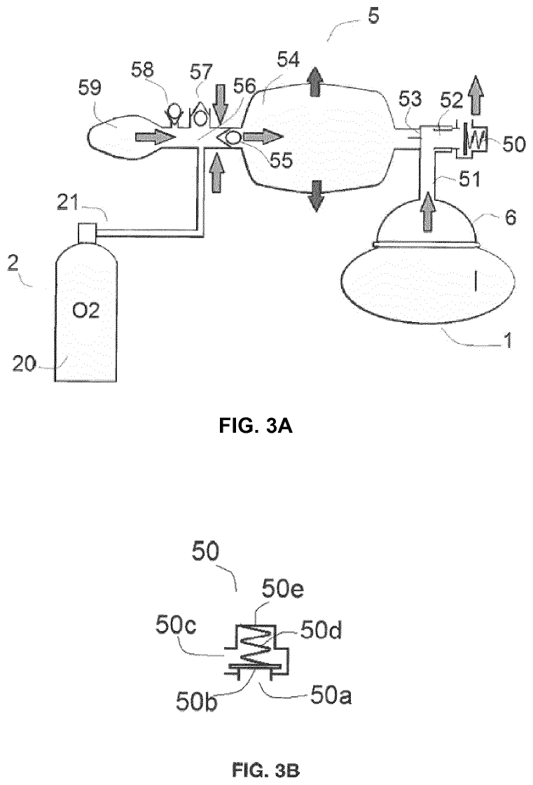

FIG. 3A shows an expiration phase of the commercially available resuscitation bag 5 of FIGS. 1 and 2, when the operator has stopped squeezing the bag 54, which bag 54 enters in an expansion phase due to a negative pressure that holds back the third exhaust valve 53 thereby opening the exhalation port 52. The volume of gas accumulated in the patient's airways during the preceding inspiratory phase will travel through interface 6 and second conduit 51 before being vented to ambient atmosphere through the exhalation port 52.

The resuscitation bag 5 can also include a PEP valve 50 that creates a positive expiratory pressure, during exhalation phases, thereby helping keeping open the alveoli of the lungs of patient 1.

As detailed in FIG. 3B, such a PEP valve 50 typically comprises a spring 50d arranged in a housing 50e, which applies a constant force on a membrane 50b. The gas pressure in the PEP valve inlet port 50a, that is in fluid communication with exhalation port 52 and that applies on said membrane 50b, has to be sufficiently high for exerting a force greater than the load of the spring 50d for displacing the membrane 50b backward and opening a fluidic pathway between the inlet port 50a and an outlet port 50c of the PEP valve inlet port 50a. The fluidic pathway allows the gas pressure to escape through the outlet port 50c, thereby allowing an expiration of gas by the patient 1. It is possible to set the load of spring 50d to different expiratory pressures, such as expiratory pressures corresponding to 5 cmH₂O, 10 cmH₂O, or 20 cmH₂O.

At the same time, the negative pressure generated in bag 54 will open the second one-way valve 55 that will: i) direct the gas flow from tubing 21 into bag 54 via conduit 56, ii) empty reservoir 59 into bag 54 via conduit 56, and iii) open the air admission valve 57 thereby allowing ambient air entering successively into conduit 56 and bag 54, as shown in FIG. 3A.

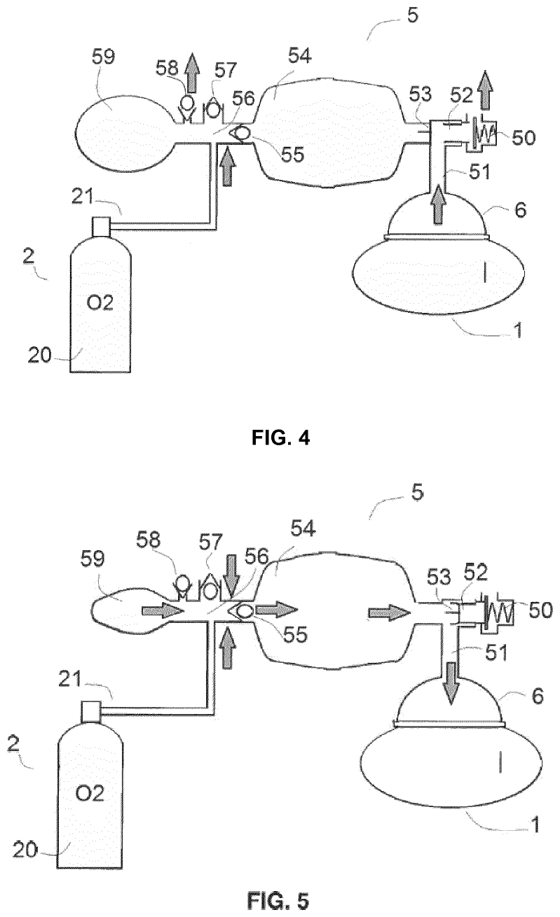

Further, FIGS. 4-6 show a sequence of thoracic compressions (TC) in association with the resuscitation bag 5 of FIGS. 1, 2 and 3A.

In FIG. 4, the resuscitation bag 5 is represented in its “rest” state, i.e. not active state, for example as it is before being used. The gas bag 54 and reservoir 59 are filled with gas and ready for an insufflation. The oxygen flowing from cylinder 20 and tubing 21 enters conduit 56 and is vented to the atmosphere through the first exhaust valve 58. When the bag 54 is in its “rest” state, the operator usually starts to exert thoracic compressions or TCs on the patient 1. Due to the TCs, the third exhaust valve 53 is pushed back, i.e. closed, thereby occluding the fluidic pathway 52 between gas bag 54 and second conduit 51. Indeed, a TC expels a small volume of gas from the patient's airways which travels backwardly through second conduit 51, exhaust port 52, and PEP valve 50. Actually, PEP valve 50 creates a resistance force against expired gases, which will promote or restore blood circulation in the patient's body.

When a TC is relaxed, the patient 1 enters decompression and the airway pressure becomes negative as shown in FIG. 5. The negative pressure closes PEP valve 50, i.e. occlude the fluidic passage between ports 50a and 50c (cf. FIG. 3B), and air is delivered by bag 54, thereby pushing the third exhaust valve 53 toward the exhaust port 52 for creating a fluidic passage between said gas bag 54 and conduit 51.

Meanwhile, the second one-way valve 55 allows: i) a first flow of gas, e.g. oxygen, to travel in tubing 21 and conduit 56, and ii) a second flow of gas to exit reservoir 59 and to travel in conduit 56.

Further, a third flow of gas, i.e. air, is allowed to penetrate into conduit 56 via the admission valve 57, i.e. another one-way valve. These three flows of gas enter into bag 54 thereby filling said bag 54.

However, with such architecture, several problems exist. For instance, the pressure in the patient's airways when the TC is relaxed will be equal to 0, i.e. not positive. This is clearly an issue as for providing efficient TCs, a positive pressure, such as 5 cm H₂O, is mandatory to force the alveoli of the patient to open and to improve gas exchanges.

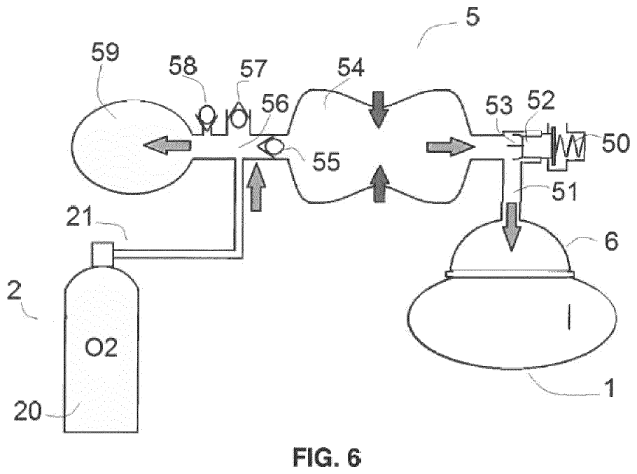

As shown in FIG. 6, when the operator performs an insufflation as described earlier, if a TC occurs during this insufflation phase, third exhaust valve 53 and second one-way valve 55 will prevent any gas exhaust. This constitutes a risk for the patient 1 as an over-pressure will appear, which can be deleterious for the lungs of the patient 1.

As shown in FIGS. 1-6, artificial resuscitation bags of the prior art do not allow to simultaneously performing safe and effective TCs and gas insufflations with the resuscitation bag. Indeed, with such known resuscitation bags, it is impossible, on the one hand, to provide TCs during insufflations phases without risking over-pressures in the lungs and, on the other hand, keep a positive pressure during decompression phases, which negatively impacts gas exchanges and outcomes for the patient.

The present invention proposes an artificial resuscitation bag 5 that can overcome the above issues.

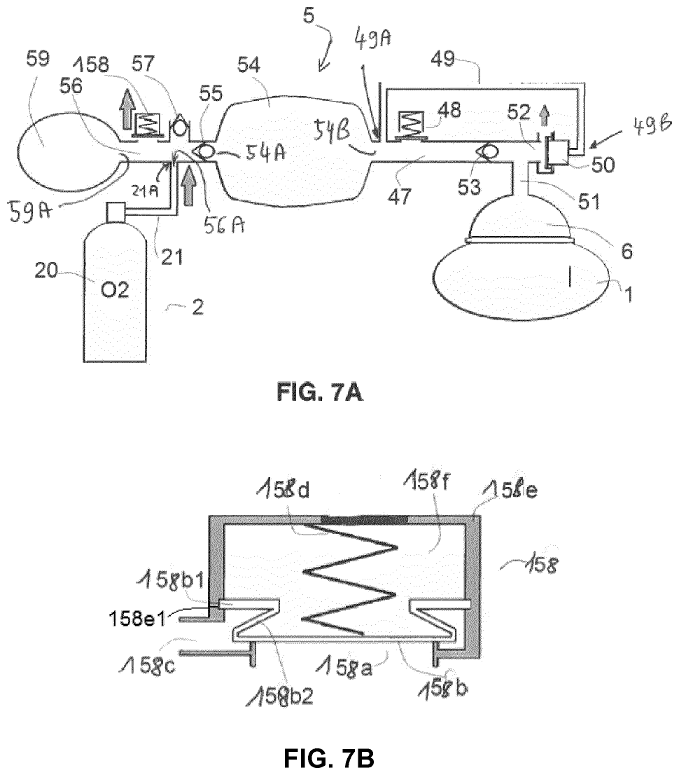

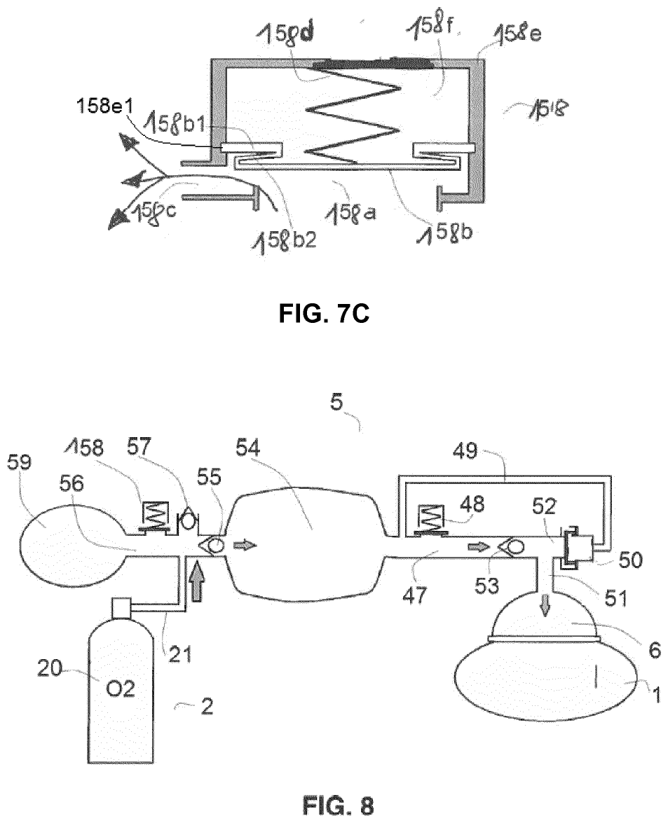

A first embodiment of an artificial resuscitation bag 5 according to the present invention is shown in FIGS. 7-13, whereas a second embodiment of an artificial resuscitation bag 5 according to the present invention is shown in FIG. 12.

FIG. 7A shows a first embodiment of a resuscitation bag 5 according to the present invention, allowing TCs to be performed while insufflating gas, and further keeping the patient's airways at a positive pressure level, i.e. greater than 0.

The artificial resuscitation bag 5 of FIGS. 7-12 has roughly the same architecture as the bag of FIG. 1-6. It comprises a deformable bag 54 comprising a gas inlet 54A and a gas outlet 54B, a gas reservoir 59 comprising an outlet orifice 59A, a first conduit element 56 fluidly connected to the outlet orifice 59A of the gas reservoir 59 and to the gas inlet 54A of the deformable bag 54, a first one-way admission valve 57 arranged in the first conduit element 56 and fluidly communicating with the ambient atmosphere for allowing ambient air to enter into the first conduit element 56, and a second one-way valve 55 arranged in the first conduit element 56 between the first one-way admission valve 57 and the gas inlet 54A of the deformable bag 54 for allowing gas to travel only from the first conduit element 56 to the deformable bag 54.

Further, the artificial resuscitation bag 5 of FIG. 7A also comprises an overpressure valve 48, also called “PPEAK valve”, and a third one-way valve 53 arranged in the conduit 47 that is in fluid communication with the outlet 54B of the deformable bag 54.

The third one-way valve 53 prevents the gas to circulate backward in the conduit 47, i.e. in the direction of the deformable bag 54, whereas as the overpressure valve 48 is used for venting to the atmosphere any excess of pressure in the conduit 47, between the deformable bag 54 and the third one-way valve 53.

Furthermore, according to the present invention, the artificial resuscitation bag 5 of FIG. 7A also comprises a first PEP exhaust valve 158 (PEP=Positive Expiration Pressure) arranged in the first conduit element 56 that fluidly communicates with the ambient atmosphere for venting gas to the atmosphere, when the gas pressure, into the first conduit element 56, exceeds a given threshold, for instance a threshold pressure of about 5 cmH₂O.

In other words, the first exhaust valve 58 of FIGS. 1-6 has been replaced by the first PEP exhaust valve 158.

FIG. 7B shows a detailed embodiment of the first PEP exhaust valve 158. It comprises a spring 158d arranged in a housing 158e, which applies a constant force on a membrane 158b that corresponds to the threshold pressure of for instance about 5 cmH₂O.

The gas pressure in the inlet port 158a of the first PEP exhaust valve 158, that is in fluid communication with the first conduit element 56, and that applies on said membrane 158b, has to be sufficiently high for exerting a force greater than the load of the spring 158d for displacing the membrane 158b backward and opening a fluidic pathway between the inlet port 158a and an outlet port 158c of the first PEP exhaust valve 158, i.e. a force greater than 5 cmH₂O for instance. This allows an excessive gas pressure in the first conduit element 56 to escape to the atmosphere through the outlet port 158c of the first PEP exhaust valve 158.

The load of spring 158d has to be set at a desired threshold pressure, i.e. a given expiratory pressure, of 5 mm H₂O or greater, such as expiratory pressures corresponding to 5 cmH₂O, 10 cmH₂O, 20 cmH₂O or 30 cmH₂O.

The deformable membrane 158b is tightly attached by its lips 158b1 to one or several grooves 158e1 arranged in the rigid structure forming the control valve housing 158e of the first PEP exhaust valve 158. A deformable portion 158b2 of membrane 158b helps membrane 158b moving forward or backward, depending on the pressure conditions.

At rest, membrane 158b of the first PEP exhaust valve 158 prevents a fluidic connection between the inlet conduit 158a and the outlet conduit 158c, as shown in FIG. 7B, due to the force exerted by load spring 158d on membrane 158b.

FIG. 7C shows the first PEP exhaust valve 158 in its open position, when the gas pressure exceeds the threshold pressure level so that spring 158d is compressed, thereby allowing gas to escape to the atmosphere through the outlet port 158c of the first PEP exhaust valve 158.

In FIG. 7A, the resuscitation bag 5 is in an initial state or “rest” state in case of a thoracic compression. The gas reservoir 59 is filled with oxygen, the oxygen being provided by the O₂ source 2, namely the cylinder 20 delivering oxygen to reservoir 59 via an oxygen-conveying tubing 21 and first conduit element 56. The oxygen-conveying tubing 21 delivers oxygen to the first conduit element 56 through an oxygen entry 56A.

The first PEP exhaust valve 158 is opened and vents the excess of gas to the atmosphere as the gas pressure exceeds the opening threshold pressure of the first PEP exhaust valve 158 that is set at 5 cmH₂O for example. This positive pressure keeps the first one-way valve 57 closed. This pressure will be equalized in all the parts behind the second one-way valve 55, i.e. into bag 54 and subsequent components, such as conduits 47, 51 and 52.

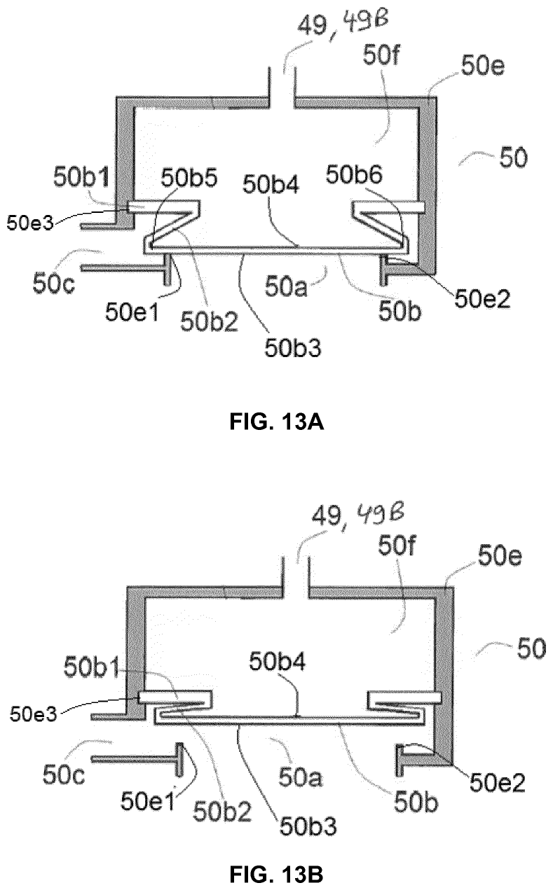

Further, the artificial resuscitation bag 5 of FIG. 7A further comprises a pneumatic control valve 50 working in differential mode as shown in FIGS. 13A and 13B. The pneumatic control valve 50 comprises a deformable membrane 50b that is tightly attached by its lips 50b1 to one or several grooves 50e3 in a rigid structure 50e, which forms the pneumatic control valve 50 housing. A deformable portion 50b2 of membrane 50b helps this membrane 50b move forward or backward, depending on the conditions. At rest, this membrane 50b prevents a fluidic connection between the inlet conduit 50a and outlet conduit 50c, as illustrated in FIG. 13A.

This is due to the fact that membrane 50b lays on edges 50e1 and 50e2 at rest, occluding inlet conduit 50a, and further a surface area difference exists between inner side 50b4 and outer side 50b3 of membrane 50b. Indeed, the inner side 50b4 of membrane 50b is delimited by extremity points 50b5 and 50b6, whereas the outer side of the membrane is defined as the diameter of inlet conduit 50a, delimited by edges 50e1 and 50e2. As a consequence, the surface of inner side 50b4 of membrane 50b is greater than the surface of outer side 50b3 of membrane 50b. Considering equal pressure on both sides of membrane 50b, a positive force gradient from inner side 50b4 to outer side 50b3 is created. The mechanical strength of membrane 50b laying on edges 50e1 and 50e2 and the positive force gradient generated by the surface difference between inner side 50b4 and outer side 50b3 of membrane 50b will define an opening pressure threshold in inlet 50a which will move membrane 50b backward to allow a fluidic connection between inlet 50a and outlet 50c, as shown in FIG. 13B. Depending on the size and characteristic of membrane 50b, an opening pressure as low as 5 mm H₂O can be set.

The pneumatic control valve 50 of FIGS. 13A and 13B further comprises a chamber 50f which is fluidically connected to a derivation conduct 49 comprising a first end 49A fluidly connected to the gas conduit 47, between the gas outlet 54B of the deformable bag 54 and the overpressure valve 48, and a second end 49B fluidly connected to the inner compartment 50f of the pneumatic valve 50, as shown in FIG. 7A. Should the derivation conduct 49 provide a positive pressure, this pressure would add a force on top of the opening pressure defined above which will in turn make it harder to open the fluidic connection between inlet 50a and outlet 50c, unless the pressure at inlet 50a follows the increase of pressure in chamber 50f, offsetting its effect.

As shown in FIG. 7A, at the very onset of a TC, the pressure in conduits 47 and 51, in derivation conduct 49 and consequently in chamber 50f of the pneumatic control valve 50 are equal and set to the PEP exhaust valve 158 value, e.g. 5cmH₂O. This means that only the opening pressure of pneumatic control valve 50 will oppose the rise of pressure resulting from the TC. Following the example set above, as soon as the pressure exceeds 5.5 cmH₂O in second conduit 51 (e.g. sum of initial PEP exhaust valve 158 value of 5 cmH₂O plus the opening pressure of 5 mmH₂O), thus closing the third one-way valve 53, the pneumatic control valve 50 will open to make a fluidic connection between inlet 50a and outlet 50c, allowing the volume expelled by the patient 1 to travel through interface 6, conduits 51 and 52, inlet 50a and exhaust port, or outlet 50c.

After a TC, follows a decompression phase as shown in FIG. 8. The pressure in the patient's airways suddenly decreases to potentially sub-atmospheric pressures. As a consequence, the flow of oxygen in first conduit element 56, coming from tubing 21, will be directed to the patient 1 to offset this decrease in pressure, opening the second one-way valve 55 and the third one-way valve 53, also called “exhalation valve”, and traveling through flexible bag 54 and conduits 47, 51 and interface 6. In addition, the pressure across the pneumatic control valve 50, which is between derivation conduct 49 and therefore chamber 50f, and conduit 52 and therefore inlet 50a will be close to 0 and as a result the pneumatic control valve 50 will be closed.

As a result a direct fluidic pathway will be created between the oxygen supply in tubing 21 and patient 1. However, the first PEP exhaust valve 158 will avoid any pressure greater that e.g. 5 cmH₂O in this fluidic pathway and will open, if necessary, to keep the pressure steady. In other words, in the phase of decompression, the patient 1 pressure airway will be kept close to e.g. 5 cm H₂O which will keep the alveoli open and enhance gas exchange.

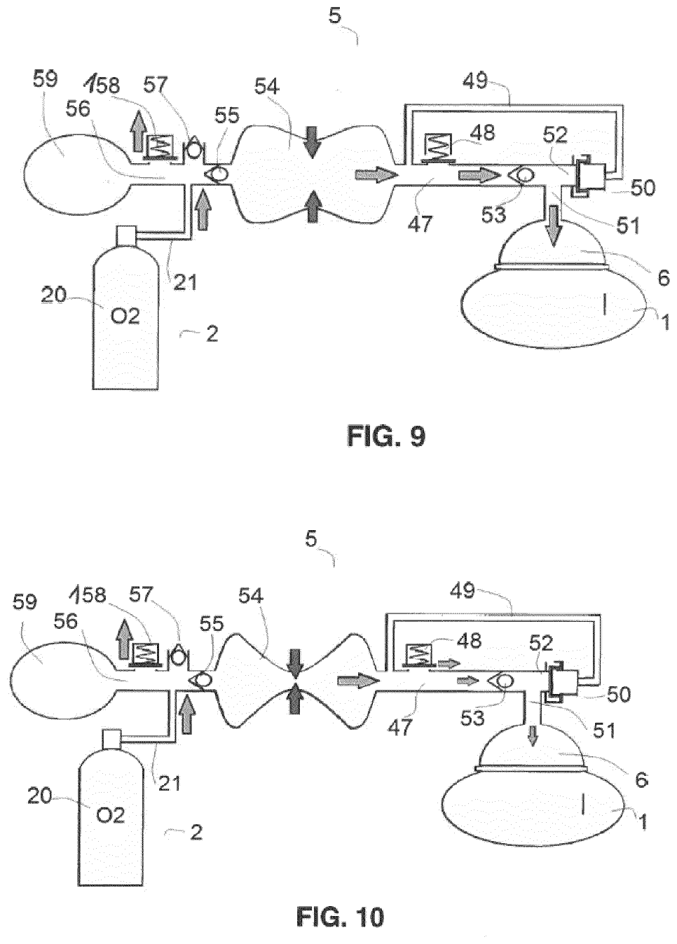

In FIG. 9, the operator starts an insufflation by squeezing the flexible bag 54, which will in turn open the third one-way valve 53. By the same mechanism, the pressure across the pneumatic control valve 50, which is between derivation conduct 49 and therefore chamber 50f, and conduit 52 and therefore inlet 50a will be close to 0. As a result, the pneumatic control valve 50 will remain closed, although the insufflation will create an increase in pressure in both sides of the pneumatic control valve 50. As a consequence, all the gas exiting the bag 54 will travel into conduits 47 and 51 and be delivered to the patient 1, via interface 6.

On the other end of the bag, such positive pressure in the flexible bag 54 will force second one-way valve 55 to close and the oxygen coming from tubing 21 and entering conduit 56 will either fill the reservoir 59 or vent to the atmosphere through the first PEP exhaust valve 158, whenever the pressure is greater than 5 cmH₂O.

At some point during the insufflations, the pressure may become too elevated. The resuscitation bag of the present invention provides a means to control this pressure as shown in FIG. 10. This function is made possible by PPEAK valve 48 which is similar to the first PEP exhaust valve 158, although its load spring is set in a way that only a pressure greater than 20 cm H₂O, for example, opens it and limits the pressure into conduits 47, 51 and patient's airways at this set value.

During the insufflation described with reference to FIGS. 9 and 10, the pneumatic control valve 50 (as shown in FIGS. 13A & 13B) assists the operator. Indeed, in case of a thoracic compression the pressure on the patient 1 side will increase, for instance above 20 cmH₂O if we consider the compression occurred while PPEAK valve 48 was limiting the pressure, and close the third one-way valve 53. This will create an imbalance in terms of pressure between conduits 51, 52 and inlet 50a and their counterpart, e.g. conduits 47, derivation conduct 49 and chamber 50f. As soon as this imbalance exceeds the opening pressure, causing a differential pressure of 5 mm H₂O, the pneumatic control valve 50 will open and make a fluidic connection between inlet 50a and outlet 50c, allowing the volume expelled by the patient 1 to travel through interface 6, conduits 51 and 52, inlet 50a and exhaust port, or outlet 50c.

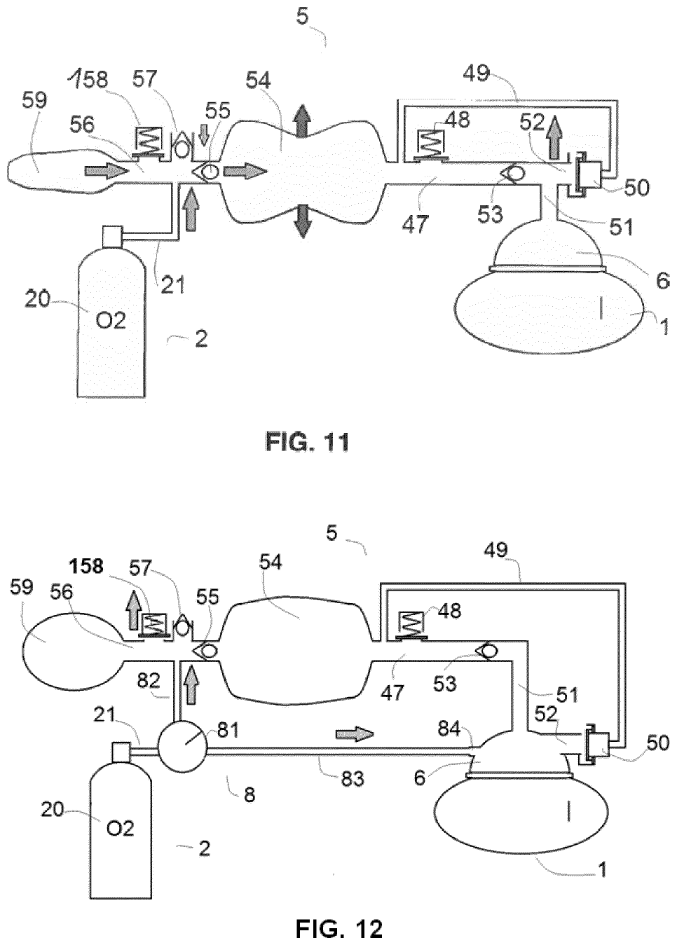

FIG. 11 shows the expiration phase, when the operator has stopped squeezing the bag 54, which enters an expansion phase. This creates a negative pressure which will open the second one-way valve 55, which will in turn: i) direct flow from tubing 21 into bag 54 via the first conduit element 56; ii) empty reservoir 59 into bag 54 via first conduit element 56; and iii) open one-way admission valve 57 which will let ambient air flow into bag 54 via conduit 56.

The same negative pressure will hold back the third one-way valve 53, close overpressure or PPEAK valve 48 and decrease the pressure in derivation conduct 49, which will in turn dramatically decrease the pressure in chamber 50f of pneumatic control valve 50. As the pressure in the patient's airways is high as a consequence of the past insufflation, the pneumatic control valve 50 opens to make a fluidic connection between inlet 50a and outlet 50c, allowing the volume expired by the patient 1 to travel through interface 6, conduits 51 and 52, inlet 50a and exhaust port, or outlet 50c. The pneumatic control valve 50 will remain open until an equilibrium is met between pressures in conduits 47 and 51, which, by virtue of the description above, should be around the pressure set by the first PEP exhaust valve 158, e.g. 5 cm H₂O and the patient 1 has returned to a low pressure level where subsequent thoracic compressions can occur, as described with reference to FIG. 7A.

The resuscitation bag 5 of the present invention has the ability to allow safe insufflations by limiting the pressure at the patient's airways while authorizing compression phases, therefore optimizing hemodynamic of the patient, and to further apply a positive pressure in the patient's airways during the thoracic decompressions to help keep the lung alveoli of the patient open and further enhance gas exchange.

A second embodiment of the resuscitation bag 5 according to the present invention that further enhances TCs, is shown in FIG. 12.

Following a TC, as shown in FIG. 7, the gas flowing into the patient 1, during the thoracic decompression phase as illustrated in FIG. 8, will partly be composed of the gas expelled from the patient 1 during TC and present in the interface 6 and conduits 51 and 52.

This gas contains a “high” level of CO₂, which replaces valuable oxygen and further prevents the CO₂ clearance from the lung.

In many cases, it is advantageous:
to lower as much as possible the space in which the CO₂ can be present, e.g. interface 6 and conduits 51 and 52, and
to “flush” out a maximum of CO₂ rich-gases, over the course of the resuscitation process.

In this aim, according to the second embodiment shown in FIG. 12, the pneumatic control valve 50 is arranged directly in the region of the interface 6 so as to be fluidly connected to interface 6 via conduit 52. Thus, pneumatic control valve 50 can more efficiently vent CO₂-enriched gases exhaled by patient 1 to the atmosphere, thereby avoiding CO₂ build-up into conduit 51. Further, an oxygen distribution system 8 comprising a by-pass line 83 and a gas distributor 81 is provided. The by-pass line 83 is arranged between the gas distributor 81 fed by the oxygen source 2 and the interface 6.

The inlet of the gas distributor 81 is fluidly connected to the oxygen source 2 via oxygen line or tubing 21. In other words, the gas distributor 81 is arranged on the oxygen line 21.

The distributor 81, when manually operated by the operator, diverts a portion of the total incoming oxygen flow either to the downstream portion 82 of the oxygen line 21, that is connected to resuscitation bag 5 via the first conduit element 56, or to the by-pass line 83 that is fluidly connected to the interface 6 via an admission port 84.

By acting on gas distributor 81, e.g. a proportional diverting valve, the operator can select/allocate the respective amounts of oxygen flowing into by-pass tubing 83 and further into the downstream portion 82 of the oxygen line 21. The first oxygen flow conveyed by the downstream portion 82 of the oxygen line 21 enters into the first conduit element 56 and, as already explained (cf. FIGS. 7 and 8), when no gas insufflation is performed, helps keep a pressure of 5 cm H₂O in the flexible bag 54 and subsequent conduit 47, derivation conduct 49 and chamber 50f, thanks to the first PEP exhaust valve 158.

Further, the second oxygen flow conveyed by the by-pass tubing 83 enters into interface 6, such as a respiratory mask, via the admission port 84. As the oxygen flow is continuous, a pressure build-up occurs in interface 6 and conduit 52 and further a pressure imbalance across pneumatic control valve 50 makes the fluidic connection between inlet conduit 50a and outlet conduit 50c to vent to the atmosphere, excessive flow, as hereinabove described in connection with FIGS. 7 to 11. Such a gas venting will also drag to the atmosphere any residual CO₂ from interface 6 and conduit 52. Vented CO₂ is substituted by fresh oxygen delivered by by-pass line 83.

The resuscitation bag 5 of the present invention constitutes a great improvement over those of the prior art.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. An artificial resuscitation bag (5) comprising:
a deformable bag (54) comprising a gas inlet (54A) and a gas outlet (54B),
a gas reservoir (59) comprising an outlet orifice (59A),
a first conduit element (56) fluidly connected to the outlet orifice (59A) of the gas reservoir (59) and to the gas inlet (54A) of the deformable bag (54),
a first one-way admission valve (57) arranged in the first conduit element (56) and fluidly communicating with an ambient atmosphere for allowing ambient air to enter into the first conduit element (56), and
a second one-way valve (55) arranged in the first conduit element (56) between the first one-way admission valve (57) and the gas inlet (54A) of the deformable bag (54) for allowing gas to travel only from the first conduit element (56) to the deformable bag (54),
and further comprising a first PEP exhaust valve (158) arranged in the first conduit element (56) and fluidly communicating with the ambient atmosphere configure and adapted for venting gas to the atmosphere when the gas pressure, into the first conduit element (56), exceeds a given threshold.

2. The artificial resuscitation bag of claim 1, wherein the opening pressure of PEP exhaust valve (158) is of at least 1 cmH₂O.

3. The artificial resuscitation bag of claim 1, wherein the opening pressure of PEP exhaust valve (158) is of between 1 cm H₂O and 30 cm H₂O

4. The artificial resuscitation bag of claim 1, wherein the first PEP exhaust valve (158) comprises a spring (158d) and a membrane (158b), said spring (158d) applying a constant force on the membrane (158b) corresponding to the threshold pressure.

5. The artificial resuscitation bag of claim 1, wherein the first PEP exhaust valve (158) comprises an inlet port (158a) in fluid communication with the first conduit element (56)

6. The artificial resuscitation bag of claim 1, wherein the first conduit element (56) comprises an oxygen entry (56A) arranged between the outlet orifice (59A) of the gas reservoir (59) and the second one-way valve (55).

7. An installation for resuscitating a person in state of cardiac arrest comprising:
an artificial resuscitation bag according to claim 1, and
an O₂ source fluidly connected to the artificial resuscitation bag by an oxygen line, configured and adapted for providing oxygen to said artificial resuscitation bag.

8. The installation of claim 7, wherein the O₂ source is fluidly connected to the oxygen entry (56A) of the first conduit element (56).
